(19) United States
(12) Patent Application Publication
(10) Pub. No.: US 2025/0010079 A1
(43) Pub. Date: Jan. 9, 2025

(54) SYSTEMS AND METHODS FOR SELECTING ELECTRODES AND PROVIDING STIMULATION

(71) Applicant: Boston Scientific Neuromodulation Corporation, Valencia, CA (US)

(72) Inventor: Hemant Bokil, Cambridge, MA (US)

(21) Appl. No.: 18/763,676

(22) Filed: Jul. 3, 2024

Related U.S. Application Data
(60) Provisional application No. 63/525,224, filed on Jul. 6, 2023.

(51) Int. Cl. A61N 1/36; A61N 1/05; A61N 1/372; A61N 1/378; G16H 20/40

(52) U.S. Cl. CPC A61N 1/36185; A61N 1/36135; G16H 20/40

(57) ABSTRACT

Method and systems are described for identifying electrodes for stimulation of a patient using a stimulation system. The stimulation system includes at least one stimulation lead implanted in a patient and having electrodes disposed thereon. The method includes obtaining a plurality of bioelectrical signals, wherein each of the bioelectrical signals is obtained using a different one, or a different combination, of the electrodes; analyzing a dataset including the bioelectrical signals to identify at least one fundamental component of the dataset, each of the at least one fundamental component identifying a contribution of one or more of the electrodes to the fundamental component; and using at least one of the at least one fundamental component to identify one or more of the electrodes for stimulation according to the contribution of each of the one or more of the electrodes to the at least one of the at least one fundamental component.

### CROSS-REFERENCE TO RELATED APPLICATIONS

This application claims the benefit under 35 U.S.C. § 119 (e) of U.S. Provisional Patent Application Ser. No. 63/525,224, filed Jul. 6, 2023, which is incorporated herein by reference.

### FIELD

The present disclosure is directed to methods and systems for stimulation of a patient. The present disclosure is also directed to methods and systems for selecting electrodes and providing stimulation of a patient.

### BACKGROUND

Implantable electrical stimulation systems have proven therapeutic in a variety of diseases and disorders. For example, deep brain stimulation systems have been used as a therapeutic modality for the treatment of Parkinson's disease, essential tremor, and the like.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator in the IPG generates electrical pulses that are delivered by the electrodes to body tissue.

### BRIEF SUMMARY

One aspect is a method for identifying electrodes for stimulation of a patient using a stimulation system. The stimulation system includes at least one stimulation lead implanted in a patient and having electrodes disposed thereon. The method includes obtaining a plurality of bioelectrical signals, wherein each of the bioelectrical signals is obtained using a different one, or a different combination, of the electrodes; analyzing a dataset including the bioelectrical signals to identify at least one fundamental component of the dataset, each of the at least one fundamental component identifying a contribution of one or more of the electrodes to the fundamental component; and using at least one of the at least one fundamental component to identify one or more of the electrodes for stimulation according to the contribution of each of the one or more of the electrodes to the at least one of the at least one fundamental component.

Another aspect is a stimulation system that includes at least one lead including a plurality of electrodes; a pulse generator coupled to the at least one lead and configured to deliver electrical energy through at least one of the electrodes of the at least one lead; a programmer for programming the pulse generator, the programmer including a memory having instructions stored thereon and a processor coupled to the memory and configured to execute the instructions to perform actions. The actions include obtaining a plurality of bioelectrical signals, wherein each of the bioelectrical signals is obtained using a different one, or a different combination, of the electrodes; analyzing a dataset including the bioelectrical signals to identify at least one fundamental component of the dataset, each of the at least one fundamental component identifying a contribution of one or more of the electrodes to the fundamental component; using at least one of the at least one fundamental component to identify one or more of the electrodes for stimulation according to the contribution of each of the one or more of the electrodes to the at least one of the at least one fundamental component; and programming the pulse generator to deliver stimulation using the one or more identified electrodes, wherein the pulse generator is configured to deliver the stimulation using the one or more identified electrodes.

Yet another aspect is a non-transitory computer readable memory having instructions stored thereon for identifying electrodes for stimulation of a patient using a stimulation system, the stimulation system including at least one stimulation lead implanted in a patient, the at least one stimulation lead including a plurality of electrodes, wherein the instructions, when executed by a processor, perform actions. The actions include obtaining a plurality of bioelectrical signals, wherein each of the bioelectrical signals is obtained using a different one, or a different combination, of the electrodes; analyzing a dataset including the bioelectrical signals to identify at least one fundamental component of the dataset, each of the at least one fundamental component identifying a contribution of one or more of the electrodes to the fundamental component; using at least one of the at least one fundamental component to identify one or more of the electrodes for stimulation according to the contribution of each of the one or more of the electrodes to the at least one of the at least one fundamental component; and programming a pulse generator to deliver stimulation using the one or more identified electrodes, wherein the pulse generator is configured to deliver the stimulation using the one or more identified electrodes.

In at least some aspects, the obtaining includes obtaining the plurality of bioelectrical signals, wherein each of the bioelectrical signals is obtained using a different one of the electrodes. In at least some aspects, the obtaining includes obtaining the plurality of bioelectrical signals, wherein a one of the bioelectrical signals is obtained for each of the electrodes of the at least one stimulation lead. In at least some aspects, the obtaining includes obtaining the plurality of bioelectrical signals, wherein each of the bioelectrical signals is a response to application of an electrical field to the patient using the stimulation system. In at least some aspects, the obtaining includes obtaining the plurality of bioelectrical signals, wherein each of the bioelectrical signals is recorded without the application of an electrical field to the patient to evoke the bioelectrical signal. In at least some aspects, the obtaining includes directing the patient to perform a particular activity and recording the plurality of bioelectrical signals during performance of the particular activity. In at least some aspects, the obtaining includes sequentially obtaining groups of the bioelectrical signals, wherein each of the groups includes a plurality of the bioelectrical signals obtained simultaneously.

In at least some aspects, the analyzing includes decomposing the dataset. In at least some aspects, the decomposing includes computing a cross-spectral matrix of the dataset. In at least some aspects, the method or actions further include determining a plurality of eigenvalues and eigenvectors of a matrix including the dataset, wherein the fundamental components include the eigenvectors.

In at least some aspects, the analyzing includes analyzing the dataset to identify a plurality of the fundamental components of the dataset, wherein the using includes using a plurality of the fundamental components to identify one or more of the electrodes for stimulation according to the contribution of each of the one or more of the electrodes to the plurality of the fundamental components. In at least some aspects, the using includes using at least one of the fundamental components to identify a plurality of the electrodes for stimulation according to the contribution of each of the electrodes of the plurality of electrodes to the at least one of the fundamental components. In at least some aspects, the method or the actions further include determining a fractionalization of the identified electrodes according to the contribution of each of the identified electrodes to the at least one of the fundamental components.

In at least some aspects, the using includes selecting a frequency based on concentration of energy in the one of the at least one fundamental component. In at least some aspects, the using includes identifying one or more of the fundamental components meeting a requirement of a threshold amount of a concentration of energy, wherein the identified one or more of the fundamental components are used for the identification of the one or more of the electrodes for stimulation. In at least some aspects, the using includes identifying the one or more of the electrodes for stimulation with a requirement of a threshold amount of contribution to the at least one of the at least one fundamental component.

In at least some aspects, the method or the actions further include programming a pulse generator to deliver stimulation using the one or more identified electrodes and delivering electrical stimulation using the pulse generator and the one or more identified electrodes.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes one or more leads that can be coupled to an IPG;

FIG. 2 is a block diagram of elements of an electrical stimulation system;

FIG. 3A is a schematic perspective view of a distal portion of one embodiment of an electrical stimulation lead with segmented electrodes;

FIG. 3B is a schematic perspective view of a distal portion of another embodiment of an electrical stimulation lead with segmented electrodes;

FIG. 3C is a schematic perspective view of a distal portion of a third embodiment of an electrical stimulation lead with segmented electrodes;

FIG. 3D is a schematic perspective view of a distal portion of a fourth embodiment of an electrical stimulation lead with segmented electrodes;

FIG. 3E is a schematic perspective view of a distal portion of a fifth embodiment of an electrical stimulation lead with segmented electrodes;

FIG. 4 is a flowchart of one embodiment of a method for identifying electrodes for stimulation of a patient using a stimulation system; and

FIG. 5 is a flowchart of another embodiment of a method for identifying electrodes for stimulation of a patient using a stimulation system.

### DETAILED DESCRIPTION

The present disclosure is directed to methods and systems for stimulation of a patient. The present disclosure is also directed to methods and systems for selecting electrodes and providing stimulation of a patient.

Implantable electrical stimulation systems and devices are used herein to exemplify the inventions, but it will be understood that these inventions can be utilized with other stimulation or modulation systems and devices. Examples of implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties. Examples of electrical/optical stimulation systems, which include one or more optical emitters in addition to electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

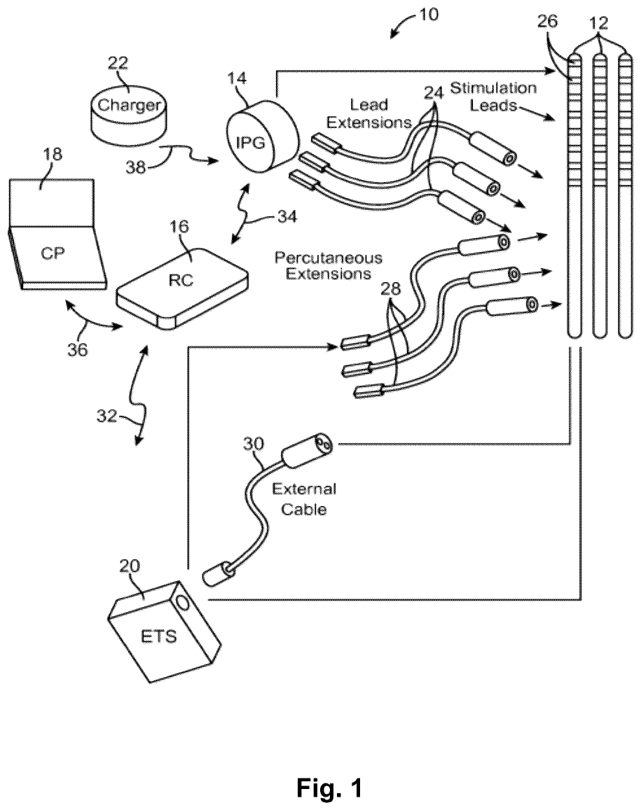

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameter values. The implantable pulse generator can be implanted into a patient's body, for example, below the patient's clavicle area or within the patient's abdominal cavity or at any other suitable site. The implantable pulse generator 14 can have multiple stimulation channels which may be independently programmable to control the magnitude of the current stimulus from each channel. In some embodiments, the implantable pulse generator 14 can have any suitable number of stimulation channels including, but not limited to, 4, 6, 8, 12, 16, 32, or more stimulation channels. The implantable pulse generator 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameter values. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14, for example, to be turned on or off and to be programmed with different stimulation parameter sets. The IPG 14 may also be operated to modify the programmed stimulation parameter values to actively control the characteristics of the electrical stimulation energy output by the IPG 14. In at least some embodiments, the CP 18 (or RC 16 or other programming device) allows a user, such as a clinician, the ability to program stimulation parameter values for the IPG 14 and ETS 20 in the operating room and in follow-up sessions. Alternately, or additionally, in at least some embodiments, stimulation parameter values can be programed via wireless communications (e.g., Bluetooth) between the RC 16 (or other external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). In at least some embodiments, the stimulation parameter values provided by the CP 18 are also used to program the RC 16, so that the stimulation parameter values can be subsequently modified by operation of the RC 16 in a stand-alone mode (i.e., without the assistance of the CP 18). The CP 18 or RC 16 can be any suitable device including, but not limited to, a computer or other computing device, laptop, mobile device (for example, a mobile phone or tablet), or the like or any combination thereof. The CP 18 or RC 16 can include software applications for interacting with the IPG 14 or ETS 20 and for programming the IPG 14 or ETS 20.

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

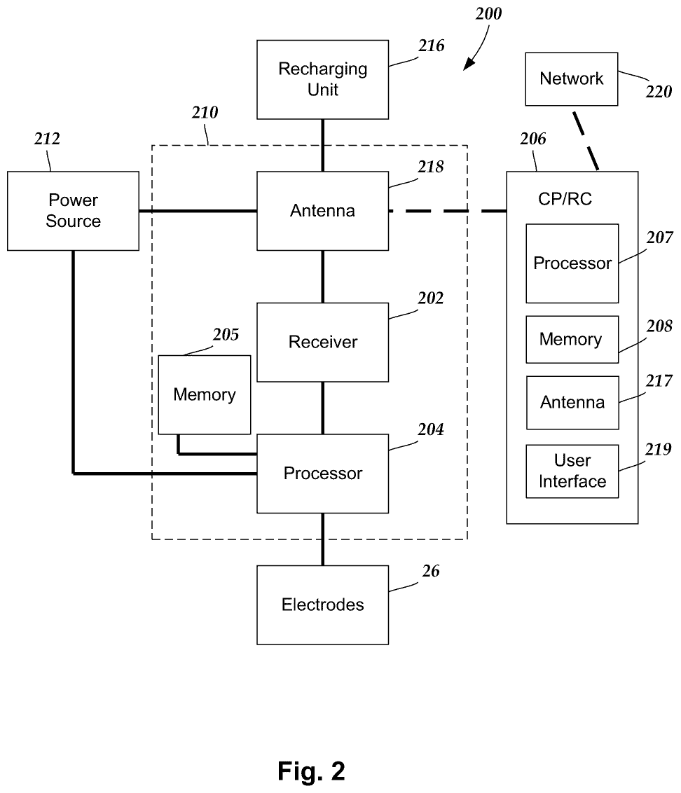

FIG. 2 is a schematic overview of one embodiment of components of an electrical stimulation system 200 including an electronic subassembly 210 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 212, antenna 218, receiver 202, processor 204, and memory 205. Some of the components (for example, power source 212, antenna 218, receiver 202, processor 204, and memory 205) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 206, can include a processor 207, memory 208, an antenna 217, and a user interface 219. The user interface 219 can include, but is not limited to, a display screen on which a digital user interface can be displayed and any suitable user input device, such as a keyboard, touchscreen, mouse, track ball, or the like or any combination thereof.

Any power source 212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the antenna 218 or a secondary antenna. The external power source can be in a device that is mounted on the skin of the user or in a unit that is provided near the user on a permanent or periodic basis.

If the power source 212 is a rechargeable battery, the battery may be recharged using the antenna 218, if desired. Power can be provided to the battery for recharging by inductively coupling the battery through the antenna to a recharging unit 216 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 204 is generally included to control the timing and electrical characteristics of the electrical stimulation system. For example, the processor 204 can, if desired, control one or more of the timing, frequency, amplitude, width, and waveform of the pulses. In addition, the processor 204 can select which electrodes can be used to provide stimulation, if desired. In some embodiments, the processor 204 may select which electrode(s) are cathodes and which electrode(s) are anodes. In some embodiments, the processor 204 may be used to identify which electrodes provide the most useful stimulation of the desired tissue. Instructions for the processor 204 can be stored on the memory 205. Instructions for the processor 207 can be stored on the memory 208.

Any processor 204 can be used for the IPG and can be as simple as an electronic device that, for example, produces pulses at a regular interval or the processor can be capable of receiving and interpreting instructions from the CP/RC 206 (such as CP 18 or RC 16 of FIG. 1) that, for example, allows modification of pulse characteristics. In the illustrated embodiment, the processor 204 is coupled to a receiver 202 which, in turn, is coupled to the antenna 218. This allows the processor 204 to receive instructions from an external source to, for example, direct the pulse characteristics and the selection of electrodes, if desired. Any suitable processor 207 can be used for the CP/RC 206.

Any suitable memory 205, 208 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 218 is capable of receiving signals (e.g., RF signals) from an antenna 217 of a CP/RC 206 (see, CP 18 or RC 16 of FIG. 1) which is programmed or otherwise operated by a user. The signals sent to the processor 204 via the antenna 218 and receiver 202 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 200 to cease operation, to start operation, to start signal acquisition, to stop signal acquisition, to start charging the battery, or to stop charging the battery. In other embodiments, the stimulation system does not include an antenna 218 or receiver 202 and the processor 204 operates as programmed.

Optionally, the electrical stimulation system 200 may include a transmitter (not shown) coupled to the processor 204 and the antenna 218 for transmitting signals back to the CP/RC 206 or another unit capable of receiving the signals. For example, the electrical stimulation system 200 may transmit signals indicating whether the electrical stimulation system 200 is operating properly or not or indicating when the battery needs to be charged or the level of charge remaining in the battery. The processor 204 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 220, such as a local area network, wide area network, the Internet, or the like or any combination thereof.

At least some of the stimulation electrodes can take the form of segmented electrodes that extend only partially around the perimeter (for example, the circumference) of the lead. These segmented electrodes can be provided in sets of electrodes, with each set having electrodes circumferentially distributed about the lead at a particular longitudinal position.

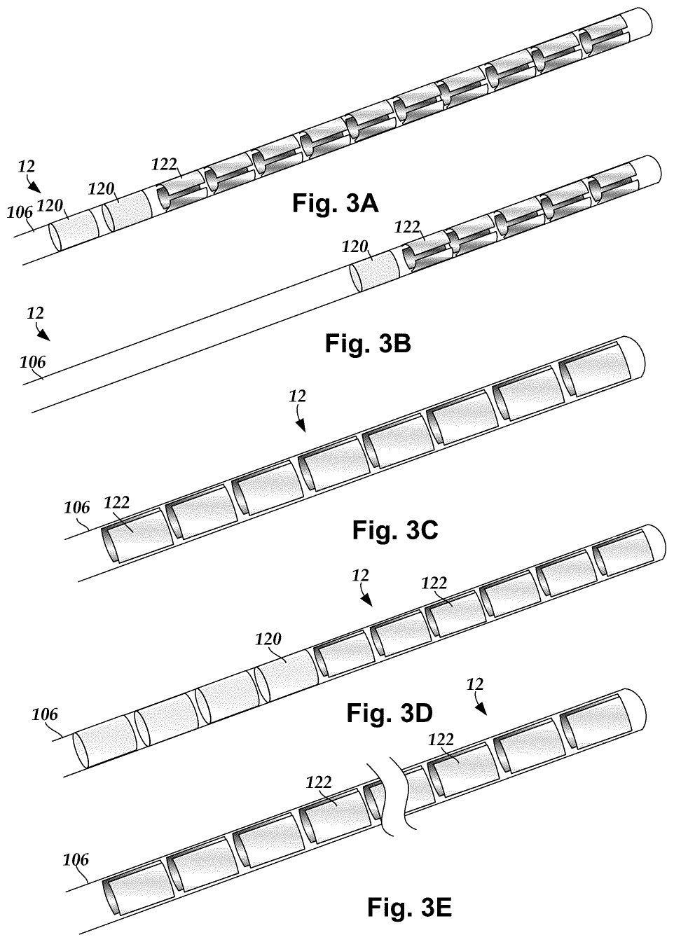

In FIGS. 3A, 3B, and 3D the electrodes are shown as including both ring electrodes 120 and segmented electrodes 122. In some embodiments, the electrodes are all segmented electrode 122, as illustrated in FIGS. 3C and 3E. The segmented electrodes 122 of FIG. 3A are in sets of three, where the three segmented electrodes of a particular set are electrically isolated from one another and are circumferentially offset along the lead 12. Any suitable number of segmented electrodes can be formed into a set including, for example, two, three, four, or more segmented electrodes. The lead 12 of FIG. 3A has thirty segmented electrodes 122 (ten sets of three electrodes each) and two ring electrodes 120 for a total of 32 electrodes.

Segmented electrodes can be used to direct stimulus current to one side, or even a portion of one side, of the lead. When segmented electrodes are used in conjunction with an implantable pulse generator that delivers current stimulus, current steering can be achieved to deliver the stimulus more precisely to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue.

FIG. 3A illustrates a 32-electrode lead 12 with a lead body 106 and two ring electrodes 120 proximal to thirty segmented electrodes 122 arranged in ten sets of three segmented electrodes each. In the illustrated embodiments, the ring electrodes 120 are proximal to the segmented electrodes 122. In other embodiments, the ring electrodes 120 can be proximal to, or distal to, or any combination thereof.

Any number of segmented electrodes 122 may be disposed on the lead body including, for example, one, two, three, four, five, six, seven, eight, nine, ten, eleven, twelve, thirteen, fourteen, fifteen, sixteen, twenty, twenty-four, twenty-eight, thirty, thirty-two, or more segmented electrodes 122. It will be understood that any number of segmented electrodes 122 may be disposed along the length of the lead body. A segmented electrode 122 typically extends only 75%, 67%, 60%, 50%, 40%, 33%, 25%, 20%, 17%, 15%, or less around the circumference of the lead.

The segmented electrodes 122 may be grouped into sets of segmented electrodes, where each set is disposed around a circumference of the lead 12 at a particular longitudinal portion of the lead 12. The lead 12 may have any number of segmented electrodes 122 in a given set of segmented electrodes. The lead 12 may have one, two, three, four, five, six, seven, eight, or more segmented electrodes 122 in a given set. The lead 12 may have any number of sets of segmented electrode including, but not limited to, one, two, three, four, five, six, eight, ten, twelve, fifteen, sixteen, twenty, or more sets. The segmented electrodes 122 may be uniform, or vary, in size and shape. In some embodiments, the segmented electrodes 122 are all of the same size, shape, diameter, width or area or any combination thereof. In some embodiments, the segmented electrodes 122 of each circumferential set (or even all segmented electrodes disposed on the lead 12) may be identical in size and shape.

Each set of segmented electrodes 122 may be disposed around the circumference of the lead body to form a substantially cylindrical shape around the lead body. The spacing between individual electrodes of a given set of the segmented electrodes may be the same, or different from, the spacing between individual electrodes of another set of segmented electrodes on the lead 12. In at least some embodiments, equal spaces, gaps, or cutouts are disposed between each segmented electrode 122 around the circumference of the lead body. In other embodiments, the spaces, gaps, or cutouts between the segmented electrodes 122 may differ in size or shape. In other embodiments, the spaces, gaps, or cutouts between segmented electrodes 122 may be uniform for a particular set of the segmented electrodes 122, or for all sets of the segmented electrodes 122. The sets of segmented electrodes 122 may be positioned in irregular or regular intervals along a length of the lead body.

The electrodes of the lead 12 are typically disposed in, or separated by, a non-conductive, biocompatible material of a lead body 106 including, for example, silicone, polyurethane, and the like or combinations thereof. The lead body 106 may be formed in the desired shape by any process including, for example, extruding, molding (including injection molding), casting, and the like. Electrodes and connecting wires can be disposed onto or within a lead body either prior to or subsequent to a molding or casting process. The non-conductive material typically extends from the distal end of the lead body 106 to the proximal end of the lead body 106.

FIG. 3B to 3E illustrate other embodiments of leads with segmented electrodes 122. FIG. 3B illustrates a sixteen electrode lead 12 having one ring electrode 120 that is proximal to five sets of three segmented electrodes 122 each. FIG. 3C illustrates a sixteen electrode lead 12 having eight sets of two segmented electrodes 122 each. As illustrated in FIG. 3C, an embodiment of a lead 12 does not necessarily include a ring electrode. FIG. 3D illustrates a sixteen electrode lead 12 having four ring electrodes 120 that are proximal to six sets of two segmented electrodes 122 each. FIG. 3E illustrates a thirty-two electrode lead 12 having sixteen sets of two segmented electrodes 122 each (for clarity of illustration, not all of the electrodes are shown). It will be recognized that any other electrode combination of ring electrodes, segmented electrodes, or both types of electrodes can be used.

When the lead 12 includes both ring electrodes 120 and segmented electrodes 122, the ring electrodes 120 and the segmented electrodes 122 may be arranged in any suitable configuration. For example, when the lead 12 includes two or more ring electrodes 120 and one or more sets of segmented electrodes 122, the ring electrodes 120 can flank the one or more sets of segmented electrodes 122. Alternately, the two or more ring electrodes 120 can be disposed proximal to the one or more sets of segmented electrodes 122 or the two or more ring electrodes 120 can be disposed distal to the one or more sets of segmented electrodes 122.

The electrodes 120, 122 may have any suitable longitudinal length including, but not limited to, 2, 3, 4, 4.5, 5, or 6 mm. The longitudinal spacing between adjacent electrodes 120, 122 may be any suitable amount including, but not limited to, 1, 2, or 3 mm, where the spacing is defined as the distance between the nearest edges of two adjacent electrodes. In some embodiments, the spacing is uniform between longitudinally adjacent electrodes along the length of the lead. In other embodiments, the spacing between longitudinally adjacent electrodes may be different or non-uniform along the length of the lead.

Examples of leads with segmented electrodes include U.S. Patent Application Publications Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/0197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

It will be understood that any suitable type of stimulation can be performed using the systems and method described herein including, but not limited to, deep brain stimulation, spinal cord stimulation, vagal stimulation, peripheral nerve stimulation, or the like or any combination thereof.

Conventionally, the selection of one or more electrodes, as well as other stimulation parameters, for effective stimulation often includes individually testing electrodes or sets of electrodes and observing effect(s) (one or more therapeutic effects or side effects or any combination thereof) of the stimulation. This conventional procedure can be time consuming and may be further lengthened in circumstances when the stimulation effect(s) may require observation over a period of time for detection. There is a need for alternative or supplemental methods for identifying one or more electrodes for stimulation.

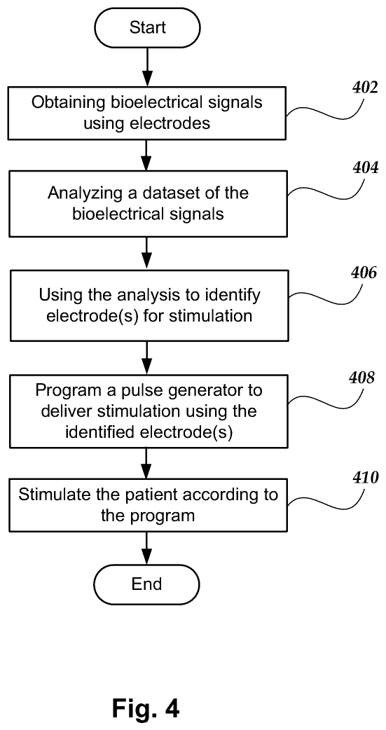

As an alternative or in addition to this conventional procedure, joint analysis of multiple bioelectrical signals obtained from the electrodes can be used to identify one or more electrodes for stimulation or for facilitating a process for selecting one or more electrodes for stimulation. FIG. 4 illustrates one embodiment of a method for identifying electrodes for stimulation of a patient using a stimulation system having at least one stimulation lead with electrodes disposed thereon. In step 402, bioelectrical signals are obtained (for example, sensed) using the electrodes. In at least some embodiments, each of the bioelectrical signals (for example, N bioelectrical signals) is obtained from one or more electrodes over a period of time (for example, T time points). In at least some embodiment, bioelectrical signals are obtained from each of the electrodes of the lead(s) or from a subset of those electrodes. Any number of bioelectrical signals can be obtained. In at least some embodiments, at least two, three, four, six, eight, ten, twelve, sixteen, twenty, or more (or any other suitable number of) bioelectrical signals are obtained.

In at least some embodiments, each of the bioelectrical signals is obtained using a different one, or a different combination, of the electrodes of the stimulation system. In at least some embodiments, a different bioelectrical signal is obtained individually for each of the electrodes or for each electrode of a subset of the electrodes of the stimulation lead(s). In at least some embodiments, each of the bioelectrical signals is obtained using a different one of the electrodes of the stimulation system.

In at least some embodiments, the bioelectrical signals are obtained simultaneously or sequentially. In at least some embodiments, the bioelectrical signals are obtained sequentially in groups of two or more bioelectrical signals, where each group of bioelectrical signals are obtained simultaneously. Any number of groups can be used including, but not limited to, two, three, four, six, eight, or more (or any other suitable number of) groups. As an example, for a lead with sixteen electrodes and for obtaining an individual bioelectrical signal for each of the electrodes, four groups of bioelectrical signals can be obtained sequentially with the four bioelectrical signals of each group being obtained simultaneously.

In at least some embodiments in which the bioelectrical signals are not all obtained simultaneously, the individual bioelectrical signals or groups of bioelectrical signals are obtained at different periods of time and under conditions that are intended to be the same or similar. In at least some embodiments, the bioelectrical signals are obtained in response to application of an electrical field (for example, the same electrical field for each of the bioelectrical signals) to the patient. For example, the electrical field is applied to the patient through one or more of the electrodes and the bioelectrical signals are recorded in response to the application of the electrical field to the patient. Examples of such bioelectrical signals include, but are not limited to, an evoked potential (EP), an evoked compound action potential (ECAP), an evoked resonant neural activity (ERNA), or the like or any combination thereof.

In at least some embodiments, each of the bioelectrical signals is a bioelectrical signal recorded without the application of an electrical field to the patient. Examples of such bioelectrical signals can include, but are not limited to, a local field potential (LFP), an electroencephalogram (EEG), an electrocardiogram (ECG), an electrocorticogram (ECOG), an electromyogram (EMG), or the like or any combination thereof.

In at least some embodiments, the signals obtained from the electrodes can include signals generated during undirected or directed activity of the patient. In at least some embodiments, the patient is directed to perform a particular activity and each of the bioelectrical signals is recorded during or after the performance of the activity. This may be repeated multiple times to record multiple bioelectrical signals or groups of bioelectrical signals. In at least some other embodiments, each of the bioelectrical signals is recorded without any specific direction to the patient or without applying an electrical field or both.

In step 404, all or a subset of the bioelectrical signals form a dataset and that dataset is analyzed. In at least some embodiments, the analysis can be performed in the time domain using the bioelectrical signals or after conversion (e.g., via Fourier transformation of the bioelectrical signals) to the frequency domain. Any suitable, known time-domain or frequency-domain technique (or any combination of such techniques) can be used for the analysis.

In at least some embodiments, to analyze the dataset, the bioelectrical signals (for example, represented by sensed signals at a sequential set of time points) are arranged as a matrix with each row or column corresponding to one of the bioelectrical signals. In at least some embodiments, the dataset can be viewed as a multi-dimensional matrix N×T (or T×N), where N is the number of bioelectrical signals (in at least some embodiments, the number of electrodes) and T is the number of time points recorded for the bioelectrical signals.

Recorded brain activity (or other signals) can be thought of as a signal of interest (for example, a neural signal) superimposed with noise. Activity recorded from different electrodes is necessarily unique to the particular recording electrode. There can be commonalities between the bioelectrical signals recorded at different electrodes. Fundamental mode decomposition can be used to extract, from multiple recorded signals, the portions that are considered most relevant. Examples of ways for extracting these portions include, but are not limited to, principle component analysis (PCA), independent component analysis (ICA), graph theoretic techniques, or the like or any combination thereof. Each method may emphasize different aspects of the bioelectrical signals. For example, ICA emphasizes statistical independence of the modes, PCA emphasizes orthogonality and capturing the variance in the data, etc. In at least some embodiments, the analysis takes data from many electrodes and replaces that data with one, two, three, four, or more few modes. Each mode contains a contribution from one or more of the electrodes. The electrode contributions can be used to determine fractionalization for the stimulation.

In at least some embodiments, the dataset of bioelectrical signals is decomposed to identify one or more fundamental components. The number of fundamental components that are determined can be one, two, three, four, five, six, or more fundamental components. In at least some embodiments, at least some of the fundamental components representing lower energy fundamental components may be designated or treated as noise. In at least some embodiments, the decomposition may elucidate synergistic effects for using multiple electrodes. In at least some embodiments, the decomposition includes computing a cross-spectral matrix of the dataset matrix.

Any suitable frequency-domain or time-domain technique can be used to decompose the dataset into fundamental components. Other analytical techniques include, but are not limited to, coherence, global coherence, independent components analysis, graph theoretic calculations, or the like or any combination thereof.

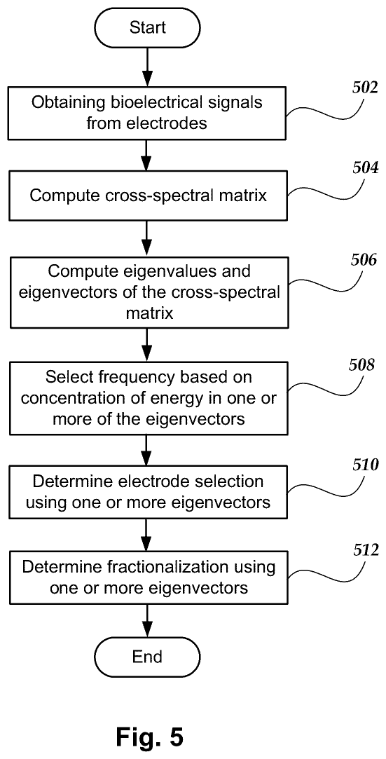

As an example, methods for analyzing the data set include frequency-domain or time-domain singular value decomposition. FIG. 5 illustrates one embodiment of these techniques for analysis. The steps in FIG. 5 correspond to steps 402 to 406 of FIG. 4 In at least some embodiments, in step 502 bioelectrical signals X(t,n) are obtained where t is the time point ranging from 1 to T and n is the signal index running from 1 to N, where N is the number of bioelectrical signals (in at least some embodiments, the number of electrodes) and T is the number of time points recorded for the bioelectrical signals. In step 504, a cross-spectral matrix is computed: Cij(f)=X(f,i)X*(f,j) where X(f,i) is an estimate of the Fourier Transform of X(t,i).

In step 506, eigenvalues of the cross-spectral matrix for at least one frequency of interest (for example, a beta frequency of brainwaves) are computed using, for example, a multi-taper method or any other suitable technique. This results in eigenvalues λ₁(f)>λ₂(f)> . . . >λ_{N}(f) and corresponding eigenvectors v₁(f), v₂(f), . . . , v_{N}(f). This decomposition diagonalizes the cross-spectral matrix (i.e., transforms the cross-spectral matrix into a new basis where the off-diagonal terms of the cross-spectral matrix are zero). The eigenvectors v₁(f), v₂(f), . . . , v_{N}(f) are uncorrelated at this frequency and the diagonal terms indicate the amount of the total energy is contained in each eigenvector. The eigenvectors are the fundamental components of the matrix of bioelectrical signals. In at least some embodiments, the eigenvectors, eigenvalues, diagonalized matrix, or any combination thereof may be displayed on a programming device, such as the RC16 or CP 18. In at least some embodiments, the eigenvectors are determined using the singular value decomposition technique.

Returning to FIG. 4, in step 406, the analysis is used to identify one or more electrodes for stimulation. As an example, the one or more of the fundamental components determined by the techniques described above can be used to identify electrode(s) for stimulation. In at least some embodiments, one or more of the fundamental components (for example, one, two, three, four, five, or six or more of the fundamental components) can be selected for the identification of electrode(s).

In at least some embodiments, the selection of one or more electrodes and the fractionalization of the electrodes can be based on one or more of the fundamental components (i.e., the eigenvectors). As an example, in step 508 of FIG. 5, a frequency can be selected at which the concentration of energy in the leading eigenvector is (or selected set of eigenvectors are) the highest (e.g., λ₁(f)/[λ₁(f)+ . . . +λ_{N}(f)]—this ratio is called the global coherence). In at least some embodiments, the leading eigenvector or the selected set of eigenvectors is used to guide electrode choice for the stimulation.

In step 510, the non-zero components of the eigenvector indicate the electrode selection. In at least some embodiments, if a component of the eigenvector is less than a threshold value, sₜ, then the electrode is not selected because the contribution of the component is too small.

In step 512, the fractionalization is equal to the proportion of each of the components of the leading eigenvector (or a combination of two or more eigenvectors) of the sum of the components. The fractionalization is determined using the components that only correspond to the selected electrodes.

As an alternative to steps 510 and 512, a single electrode (or a set of electrodes) with the largest contribution to the selected eigenvector(s) is selected for stimulation. The entire stimulation amplitude is presented at the selected electrode (or set of electrodes).

As another alternative to steps 510 and 512, the eigenvectors can be input into an algorithm that determines the electrode selection, fractionalization, or both. In at least some embodiments, the algorithm is a machine learning algorithm that uses a training set of stimulation instances and eigenvectors corresponding to the stimulation instances.

For multi-site stimulation (C stimulation sites which may be stimulated simultaneously or in any suitable sequence or any combination thereof), the selection of one or more electrodes and the fractionalization of the electrodes for each of the C stimulation sites can be based on the components of a corresponding eigenvector. In at least some other embodiments, as many eigenvectors (for example, K eigenvectors) are selected as needed to capture a selected percentage of the energy (for example, 95, 90, 85, 80, 75, 60 or 50 percent or any other suitable percentage). The combination of these K eigenvectors are then used to identify electrodes and fractionalization for stimulation fields.

In at least some embodiments, random matrix theory results can be used to determine which eigenvalues and eigenvectors are of interest and which are incoherent noise. The eigenvectors can then be used to select electrode(s) and fractionalization(s). In at least some embodiments, a threshold is used to determine which eigenvectors to retain.

As one example of the steps illustrated in FIG. 5 using a lead with four electrodes, an individual bioelectrical signal is obtained from each of the electrodes—X(t,n) are obtained where t is the time point ranging from 1 to T and n is the signal index running from 1 to 4. The cross-spectral matrix is generated, as described above, and eigenvalues λ₁(f), λ₂(f), λ₃(f), and λ₄(f) and corresponding eigenvectors v₁(f), v₂(f), v₃(f), and v₄(f) are determined. For purposes of illustration, an example of a set of eigenvectors, for a selected frequency f₀, is v₁(f₀)=(a₁, b₁, c₁, 0), v₂(f₀)=(a₂, 0, c₂, 0), v₃(f₀)=(a₃, b₃, 0, 0), and v₄(f₀)=(a₄, b₄, c₄, d₄) with corresponding eigenvalues λ₁(f₀)>λ₂(f₀)>λ₃(f₀)>λ₄(f₀). In this illustrative example, a noise threshold is selected or otherwise defined as sₙ and sₙ>λ₃(f₀)>λ₄(f₀). In this illustrative example, the third and fourth eigenvectors v₃(f₀) and v₄(f₀) are discarded as the corresponding eigenvalues are below the noise threshold.

In one example, the first eigenvector v₁(f₀) is selected for determining the electrodes and fractionalization of the stimulation. In this embodiment, up to three electrodes are selected for delivering the stimulation as long as a₁, b₁, and c₁ are greater than or equal to a threshold value sₜ. If one or more of a₁, b₁, and c₁ is less than the threshold value sₜ, then that electrode is not selected. The fractionalization is the proportion of the eigenvector coefficient for the electrode over the sum of the eigenvector coefficient for all of the selected electrodes. For example, if all three of the electrodes are selected, then the fractionalization for the three electrodes is a₁/(a₁+b₁+c₁), b₁/(a₁+b₁+c₁), and c₁/(a₁+b₁+c₁) or a₁², b₁², and c₁² (where a₁²+b₁²+c₁²=1 when the eigenvectors are normalized). As another example, if c₁ is less than the threshold value sₜ, then the fractionalization for the two electrodes is a₁/(a₁+b₁) and b₁/(a₁+b₁) or, alternatively, a₁²/(a₁²+b₁²) and b₁²/(a₁²+b₁²).

In another example, both eigenvectors v₁(f₀) and v₂(f₀) are selected. Again, assuming that the combined coefficients for all three electrodes exceed the threshold value sₜ, the corresponding fractionalizations for the three electrodes is (a₁+a₂)/(a₁+b₁+c₁+a₂+c₂)), b₁/(a₁+b₁+c₁+2+c₂), and (c₁+c₂)/(a₁+b₁+c₁+a₂+c₂).

These are examples. The relationship between the contributions and the fractionalization may be more complex and may be determined experimentally or analytically or any combination thereof.

Returning to FIG. 4, in step 408, the pulse generator of the stimulation system is programmed to deliver stimulation using the one or more identified electrodes and fractionalization(s). In step 410, electrical stimulation is delivered to the patient using the pulse generator and the one or more identified electrodes and fractionalization(s).

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification and examples provide a description of the arrangement and use of the invention. Since many embodiments of the invention can be made without departing from the spirit and scope of the invention, the invention also resides in the claims hereinafter appended.

What is claimed as new and desired to be protected is:

1. A method for identifying electrodes for stimulation of a patient using a stimulation system, the stimulation system comprising at least one stimulation lead implanted in a patient, the at least one stimulation lead comprising a plurality of electrodes, the method comprising:
obtaining a plurality of bioelectrical signals, wherein each of the bioelectrical signals is obtained using a different one, or a different combination, of the electrodes;
analyzing a dataset comprising the bioelectrical signals to identify at least one fundamental component of the dataset, each of the at least one fundamental component identifying a contribution of one or more of the electrodes to the fundamental component; and
using at least one of the at least one fundamental component to identify one or more of the electrodes for stimulation according to the contribution of each of the one or more of the electrodes to the at least one of the at least one fundamental component.

2. The method of claim 1, wherein the obtaining comprises obtaining the plurality of bioelectrical signals, wherein each of the bioelectrical signals is obtained using a different one of the electrodes.

3. The method of claim 1, wherein the obtaining comprises obtaining the plurality of bioelectrical signals, wherein a one of the bioelectrical signals is obtained for each of the electrodes of the at least one stimulation lead.

4. The method of claim 1, wherein the obtaining comprises obtaining the plurality of bioelectrical signals, wherein each of the bioelectrical signals is a response to application of an electrical field to the patient using the stimulation system.

5. The method of claim 1, wherein the obtaining comprises obtaining the plurality of bioelectrical signals, wherein each of the bioelectrical signals is recorded without the application of an electrical field to the patient to evoke the bioelectrical signal.

6. The method of claim 1, wherein the obtaining comprises directing the patient to perform a particular activity and recording the plurality of bioelectrical signals during performance of the particular activity.

7. The method of claim 1, wherein the obtaining comprises sequentially obtaining groups of the bioelectrical signals, wherein each of the groups comprises a plurality of the bioelectrical signals obtained simultaneously.

8. The method of claim 1, wherein the analyzing comprises decomposing the dataset.

9. The method of claim 8, wherein the decomposing comprises computing a cross-spectral matrix of the dataset.

10. The method of claim 9, further comprising determining a plurality of eigenvalues and eigenvectors of a matrix comprising the dataset, wherein the fundamental components comprise the eigenvectors.

11. The method of claim 1, wherein the analyzing comprises analyzing the dataset to identify a plurality of the fundamental components of the dataset, wherein the using comprises using a plurality of the fundamental components to identify one or more of the electrodes for stimulation according to the contribution of each of the one or more of the electrodes to the plurality of the fundamental components.

12. The method of claim 1, wherein the using comprises using at least one of the fundamental components to identify a plurality of the electrodes for stimulation according to the contribution of each of the electrodes of the plurality of electrodes to the at least one of the fundamental components.

13. The method of claim 12, further comprising determining a fractionalization of the identified electrodes according to the contribution of each of the identified electrodes to the at least one of the fundamental components.

14. The method of claim 1, wherein the using comprises selecting a frequency based on concentration of energy in the one of the at least one fundamental component.

15. The method of claim 1, wherein the using comprises identifying one or more of the fundamental components meeting a requirement of a threshold amount of a concentration of energy, wherein the identified one or more of the fundamental components are used for the identification of the one or more of the electrodes for stimulation.

16. The method of claim 1, wherein the using comprises identifying the one or more of the electrodes for stimulation with a requirement of a threshold amount of contribution to the at least one of the at least one fundamental component.

17. The method of claim 1, further comprising programming a pulse generator to deliver stimulation using the one or more identified electrodes; and
delivering electrical stimulation using the pulse generator and the one or more identified electrodes.

18. A stimulation system, comprising
at least one lead comprising a plurality of electrodes;
a pulse generator coupled to the at least one lead and configured to deliver electrical energy through at least one of the electrodes of the at least one lead;
a programmer for programming the pulse generator, the programmer comprising a memory having instructions stored thereon and a processor coupled to the memory and configured to execute the instructions to perform actions, the actions comprising
obtaining a plurality of bioelectrical signals, wherein each of the bioelectrical signals is obtained using a different one, or a different combination, of the electrodes;
analyzing a dataset comprising the bioelectrical signals to identify at least one fundamental component of the dataset, each of the at least one fundamental component identifying a contribution of one or more of the electrodes to the fundamental component; and
using at least one of the at least one fundamental component to identify one or more of the electrodes for stimulation according to the contribution of each of the one or more of the electrodes to the at least one of the at least one fundamental component; and
programming the pulse generator to deliver stimulation using the one or more identified electrodes, wherein the pulse generator is configured to deliver the stimulation using the one or more identified electrodes.

19. The system of claim 18, wherein the using comprises using at least one of the fundamental components to identify a plurality of the electrodes for stimulation according to the contribution of each of the electrodes of the plurality of electrodes to the at least one of the fundamental components, wherein the actions further comprise determining a fractionalization of the identified electrodes according to the contribution of each of the identified electrodes to the at least one of the fundamental components.

20. A non-transitory computer readable memory having instructions stored thereon for identifying electrodes for stimulation of a patient using a stimulation system, the stimulation system comprising at least one stimulation lead implanted in a patient, the at least one stimulation lead comprising a plurality of electrodes, wherein the instructions, when executed by a processor, perform actions, the actions comprising:
obtaining a plurality of bioelectrical signals, wherein each of the bioelectrical signals is obtained using a different one, or a different combination, of the electrodes;
analyzing a dataset comprising the bioelectrical signals to identify at least one fundamental component of the dataset, each of the at least one fundamental component identifying a contribution of one or more of the electrodes to the fundamental component;
using at least one of the at least one fundamental component to identify one or more of the electrodes for stimulation according to the contribution of each of the one or more of the electrodes to the at least one of the at least one fundamental component; and
programming a pulse generator to deliver stimulation using the one or more identified electrodes, wherein the pulse generator is configured to deliver the stimulation using the one or more identified electrodes.
